(19) United States
(12) Patent Application Publication
(10) Pub. No.: US 2008/0287705 A1
(43) Pub. Date: Nov. 20, 2008

(54) METHOD OF PRODUCING ETHYNYL COMPOUND, METHOD OF HANDLING ETHYNYL COMPOUND, AND METHOD OF USING ASCORBIC ACID OR SALT THEREOF

(75) Inventor: Daisuke Urazoe, Odawara-shi (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM CORPORATION, Tokyo (JP)

(21) Appl. No.: 12/042,963

(22) Filed: Mar. 5, 2008

(30) Foreign Application Priority Data
Mar. 7, 2007 (JP) 2007-057600

(51) Int. Cl. C07C 63/16; C07C 63/64; C07C 211/44; C07C 233/65; C07C 39/18

(52) U.S. Cl. 562/480; 562/495; 564/305; 564/123; 568/716

(57) ABSTRACT

A method of producing a first ethynyl compound represented by the following formula (1), the method including reacting a second ethynyl compound represented by the following formula (2) in a liquid phase in the presence of a reducing agent to obtain the first ethynyl compound, wherein Q¹ represents an organic group; R¹ and R² each independently represent a hydrogen atom or a hydrocarbon group; and R¹ and R² may be bonded to each other.

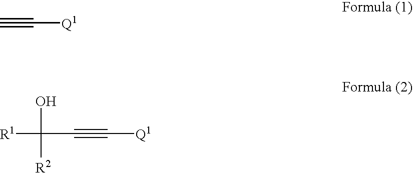

### CROSS-REFERENCE TO RELATED APPLICATION

This application claims priority under 35 USC 119 from Japanese Patent Application No. 2007-057600, the disclosure of which is incorporated by reference herein.

### BACKGROUND OF THE INVENTION

1. Field of the Invention

The present invention relates to a method of producing an ethynyl compound, a method of handling an ethynyl compound, and a method of using ascorbic acid or a salt thereof, which methods are suitable for producing, using, or handling a monosubstituted ethynyl compound, which is useful as a functional material such as a medical or agrochemical intermediate, liquid crystal, an electronic material or the like.

2. Description of the Related Art

A monosubstituted ethynyl compound is important as a functional raw material such as a medical or agrochemical intermediate, liquid crystal, an electronic material or the like. For example, as means for enhancement of heat resistance of a heat-resisting resin, 5-ethynyl isophthalic acid is disclosed (for example, refer to Japanese Patent Application Laid-Open (JP-A) No. 2002-201158). Further, an ethynyl compound is used as a partial structure of a curative medicine for hyperproliferative diseases such as cancers (for example, refer to Japanese National Phase Publication No. 2000-512990), and an ethynyl compound is used as a raw material of a compounding agent used for improvement of threshold characteristics, layer structure, orientation, and contrast ratio of a smectic liquid crystal composition (for example, refer to JP-A No. 2005-298453).

At the same time, the monosubstituted ethynyl compound has the property of being easily converted to a 1,3-diyne compound through an oxidation process. For this reason, in the reaction using a monosubstituted ethynyl compound, there have conventionally been proposed the following methods by way of example: a method in which a monosubstituted ethynyl compound is subjected to a coupling reaction with a halogenated aryl in parallel with deprotection from a state of being protected by a hydroxypropyl group (for example, refer to “Journal of Organic Chemistry”, 2001, vol. 66, pp. 1910 to 1913); and a method in which an ethynyl group terminal in a raw material is protected by an antimony compound, and the ethynyl compound is reacted and subjected to a coupling reaction with a halogenated aryl at a low temperature, thereby preventing generation of 1,4-diphenyl-1,3-butadiyne that is formed at the same time of deprotection (for example, refer to “Tetrahedron Letters” 2003, vol. 44, pp. 8589 to 8592).

### SUMMARY OF THE INVENTION

According to an aspect of the invention, there is provided a method of producing a first ethynyl compound represented by the following formula (1), the method including reacting a second ethynyl compound represented by the following formula (2) in a liquid phase in the presence of a reducing agent to obtain the first ethynyl compound:

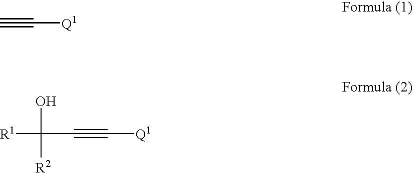

wherein Q¹ represents an organic group; R¹ and R² each independently represent a hydrogen atom or a hydrocarbon group; and R¹ and R² may be bonded to each other.

### DETAILED DESCRIPTION OF THE INVENTION

Exemplary embodiments of the present invention are as follows.
<1> A method of producing a first ethynyl compound represented by the following formula (1), the method including reacting a second ethynyl compound represented by the following formula (2) in a liquid phase in the presence of a reducing agent to obtain the first ethynyl compound:

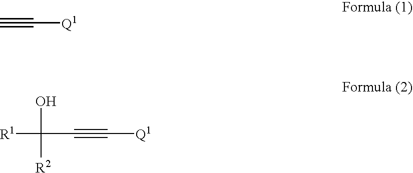

wherein Q¹ represents an organic group; R¹ and R² each independently represent a hydrogen atom or a hydrocarbon group (preferably an alkyl group having 1 to 6 carbon atoms, a cycloalkyl group having 1 to 6 carbon atoms, or an aryl group having 6 to 12 carbon atoms); and R¹ and R² may be bonded to each other.

In the method of producing an ethynyl compound described in the aforementioned item <1>, a monosubstituted ethynyl compound is produced in a state in which a compound represented by formula (2), that is a raw material, is made to exist in a liquid phase containing a reducing agent, thereby making it possible to prevent an oxidative reaction that tends to occur in the process after the monosubstituted ethynyl compound is produced. As a result, formation of a by-product (a 1,3-diyne compound) formed by an oxidation reaction that progresses after production of the ethynyl compound can be dramatically prevented. Accordingly, a monosubstituted ethynyl compound suitable for use in a functional material such as a medical or agrochemical intermediate, liquid crystal, or an electronic material can be stably produced.
<2> The method according to <1>, wherein Q¹ is a hydrocarbon group or a hetero atom-containing group, which may have a substituent.
<3> The method according to <2>, wherein Q¹ is an aromatic hydrocarbon group or an aromatic heterocycle, which may have a substituent.
<4> The method according to <3>, wherein Q¹ is an aryl group which may have a substituent.
<5> The method according to <3>, wherein Q¹ is an alkyl group which may have a substituent, an aralkyl group which may have a substituent, an alkenyl group which may have a substituent, or an alkynyl group which may have a substituent.
<6> The method according to <1>, wherein Q¹ has at least two substituents selected from the group consisting of a hydroxyl group, an amino group, an acylamino group, a carboxyl group, an acyloxy group, a sulfonyl group and phosphonyl group, and two adjacent substituents are bonded to each other to form a ring.
<7> The method according to <4>, wherein Q¹ is a phenyl group having at least one substituent, a naphthyl group having at least one substituent, or an anthranyl group having at least one substituent.
<8> The method according to <4>, wherein Q¹ is an aryl group having a substituent, and the substituent in Q¹ is a 2-carboxyl group, a 3-carboxyl group, a 3,4-dicarboxyl group, a 3,5-dicarboxyl group, a 3-amino group, a 3-acyloxy group, a 4-amino group, a 4-acylamino group, or a salt thereof.
<9> The method according to <1>, wherein the reducing agent functions so as to inhibit a dimerization reaction of the first ethynyl compound.
<10> The method according to <1>, wherein the reducing agent is at least one selected from the group consisting of dithiothreitol, β-mercaptoethanol, tartaric acid, ascorbic acid, substituted or unsubstituted hydroxylamine, and salts thereof.
<11> The method according to <10>, wherein the reducing agent is at least one selected from the group consisting of ascorbic acid, tartaric acid and salts thereof.

In the method of producing an ethynyl compound described in the aforementioned item <10>, by selecting an organic reducing agent, particularly, dithiothreitol, β-mercaptoethanol, tartaric acid, ascorbic acid, hydroxylamine or a salt thereof, the added reducing agent can be removed to a degree below the detection limit by solid-liquid separation. Accordingly, there is no case in which the usefulness of the ethynyl compound as a functional material such as a medical or agrochemical intermediate, liquid crystal or an electronic material is impaired due to a remaining reducing agent as in the case of an inorganic reducing agent. Thus, formation of a by-product (a 1,3-diyne compound) formed when the substituted ethylnyl compound is produced or used can be effectively prevented.

Further, in the method of producing an ethynyl compound described in the aforementioned item <11>, the same effects as those of the item <10> can be more effectively achieved.
<12>A method of handling an ethynyl compound, including handling a first ethynyl compound represented by the following formula (1) in the presence of a reducing agent in a liquid phase in which the first ethynyl compound is produced or used:

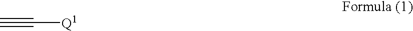

wherein Q¹ represents an organic group.

In the ethylnyl compound handling method described in the aforementioned item <12>, since the monosubstituted ethynyl compound is handled in a liquid phase containing a reducing agent, an oxidative reaction that is apt to occur in the process of handling (for example, producing or using) the monosubstituted ethynyl compound is inhibited. Therefore, formation of a by-product (a 1,3-diyne compound) can be dramatically prevented. As a result, applicability of the monosubstituted ethynyl compound as a functional material such as a medical or agrochemical intermediate, liquid crystal or an electronic material can be ensured.
<13>A method of using ascorbic acid or a salt thereof, comprising including ascorbic acid or a salt thereof in a liquid phase containing a first ethynyl compound represented by the following formula (1) to inhibit a dimerization reaction of the first ethynyl compound:

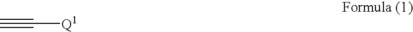

wherein Q¹ represents an organic group.

In the method of using ascorbic acid or a salt thereof described in the aforementioned item <13>, since the monosubstituted ethynyl compound is handled in a liquid phase containing a reducing agent, an oxidative reaction that is apt to occur in the handling process can be inhibited by ascorbic acid or a salt thereof. Thus, formation of a by-product (a 1,3-diyne compound) that is apt to be formed at the time of handling a monosubstituted ethynyl compound can be dramatically prevented.

The present invention can provide a method of producing an ethynyl compound, a method of handling an ethynyl compound, and a method of using ascorbic acid or a salt thereof, which is capable of preventing formation of a by-product (a 1,3-diyne compound) formed when a monosubstituted ethynyl compound is produced, used or handled.

For example, according to the present invention, a monosubstituted ethynyl compound and derivatives thereof, which are useful as a functional material such as a medical or agrochemical intermediate, liquid crystal or an electronic material, can be stably produced at a high yield, used and handled while formation of a by-product (a 1,3-diyne compound) is prevented.

A further detailed description will be hereinafter given of a method of producing an ethynyl compound, a method of handling an ethynyl compound, and a method of using ascorbic acid or a salt thereof according to the present invention.

First, a monosubstituted ethynyl compound represented by the following formula (1), a method of producing or using the same, and a method of handling the same will be described.

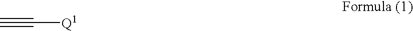

In the aforementioned formula (1), Q¹ represents an organic group. Preferably, Q¹ represents a hydrocarbon group or hetero atom-containing group, which may be unsubstituted or may have a substituent, and is more preferably an aromatic hydrocarbon group or aromatic heterocyclic group, which may be unsubstituted or may have a substituent.

Among the aforementioned groups, Q¹ is further preferably an aryl group which may be unsubstituted or may have a substituent, an alkyl group which may be unsubstituted or may have a substituent, an aralkyl group which may be unsubstituted or may have a substituent, an alkenyl group which may be unsubstituted or may have a substituent, a heterocyclic group which may be unsubstituted or may have a substituent, or an alkynyl group which may be unsubstituted or may have a substituent.

Particularly preferably, Q¹ is an aryl group which may be unsubstituted or may have a substituent, a heterocyclic group which may be unsubstituted or may have a substituent, or an alkynyl group which may be unsubstituted or may have a substituent. Most preferably, Q¹ is an aryl group which may be unsubstituted or may have a substituent. Still further preferably, Q¹ is an aryl group having a substituent.

Further, the aforementioned aryl group preferably has 5 to 16 carbon atoms in total, more preferably 6 to 12 carbon atoms in total. In the case of the aforementioned alkyl group, the total number of carbon atoms is preferably 2 to 12, and is more preferably 3 to 8. In the case of the aralkyl group, the total number of carbon atoms is preferably 6 to 18, and is more preferably, 8 to 14. In the case of the alkenyl group, the total number of carbon atoms is preferably 4 to 12, and is more preferably 5 to 8. In the case of the heterocyclic group, the total number of carbon atoms is preferably 3 to 12, and is more preferably 3 to 8. In the case of the alkynyl group, the total number of carbon atoms is preferably 4 to 12, and is more preferably 6 to 10.

Specific examples of the group represented by Q¹ in the aforementioned formula (1) include a phenyl group, a thiophenyl group, a naphthyl group, an anthranyl group, a pyrenyl group, a phthaloyl group, a phthaloylimino group, a 2-phenylethyl group, a benzoxazolyl group, a benzimdazolyl group, a cyclohexyl group, a cyclohexenyl group, an n-octyl group, an isopropenyl group, an n-hexyl group, a 5-methylhexenyl group, an isopropyl group, a sec-butyl group, a 2-phenylethylenyl group, a cyclohexenyl group, a triazinyl group, a pyrimidinyl group, a pyridinyl group, a 2-phenylethynyl group, a 1-butynyl group, and the like. Among them, a phenyl group, a naphthyl group, an anthranyl group, a triazinyl group, a pyrimidinyl group, a pyridinyl group, a thiophenyl group, a 2-phenylethynyl group, and a 1-butynyl group are preferable. More preferable are a phenyl group, a naphthyl group, and an anthranyl group.

Preferably, Q¹ is a phenyl group having at least one substituent, a naphthyl group having at least one substituent, or an anthranyl group having at least one substituent, and is more preferably a phenyl group having at least one substituent.

The aforementioned Q¹ may not be substituted or may have a substituent. Examples of a substituent in the case in which Q¹ has a substituent include a hydroxyl group, an amino group, an acylamino group, an acyloxy group, a carboxyl group, an aminocarboxyl group, a (di)alkylaminocarboxyl group, a sulfamoyl group, a (di)alkylsulfamoyl group, a cyano group, a nitro group, a sulfonyl group, an alkyl group, an alkenyl group, a phosphonyl group, an alkynyl group, and a halogen atom. Among them, a hydroxyl group, an amino group, an acylamino group, an acyloxy group, a carboxyl group, an aminocarboxyl group, a (di)alkylaminocarboxyl group, an alkyl group, an alkenyl group, an alkynyl group, and a halogen atom are preferable. More preferable are a hydroxyl group, an amino group, an acylamino group, an acyloxy group, a carboxyl group, a sulfonyl group, and a phosphonyl group.

In the foregoing, when Q¹ in formula (1) is an aryl (preferably, phenyl) group, examples of the substituent therefor include a 2-carboxyl group, a 3-carboxyl group, a 3,4-dicarboxyl group, a 3,5-dicarboxyl group, a 3-amino group, a 3-acyloxy group, a 4-amino group, a 4-acylamino group, and salts thereof.

When Q¹ has plural substituents, two of the substituents may be bonded to each other. In this case, it is preferable that plural substituents selected from the group consisting of a hydroxyl group, an amino group, an acylamino group, a carboxyl group, an acyloxy group, a sulfonyl group and a phosphoryl group may be bonded to each other to form a ring. As a preferred example, there is shown a case in which when adjacent carbon atoms are respectively substituted with a carboxyl group, these two carboxyl groups are bonded to each other to form a ring, thereby forming an acid anhydride.

Specific examples of the compound represented by the aforementioned formula (1) are shown below, but the present invention is not limited thereto.

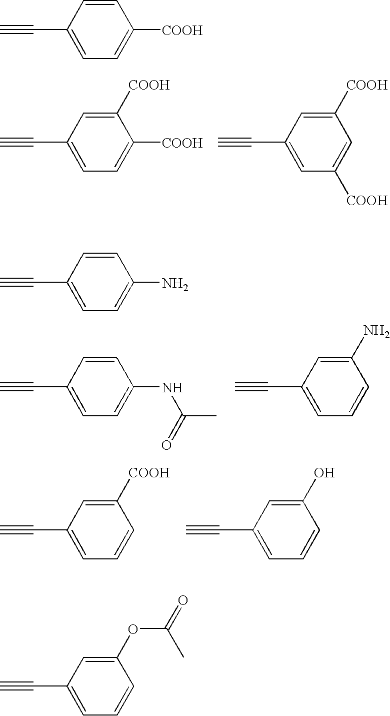

The compound represented by the aforementioned formula (1) may form a diyne compound represented by the following formula (1′) due to air, a vessel, impurities or the like.

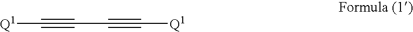

Generation of the compound represented by formula (1′) from the compound represented by formula (1) noticeably becomes fast, particularly under a basic condition in a solution, or under a gas condition. For example, the aforementioned compound is generated by stirring in the atmosphere in a protic solvent which includes alkali metal or alkali earth metal and is basic.

In the present invention, generation of the compound represented by formula (1′) from the compound represented by formula (1) can be inhibited by addition of a reducing agent.

Generation of the compound represented by formula (1′) from the compound represented by formula (1) is caused by oxidation resulting from mixing of air or unintended impurities into the compound represented by formula (1), and this oxidation process can also be estimated from the fact that the pKa of the C—H bond dissociation of a terminal ethynyl group is smaller than the pKa of the C—H bond dissociation of a vinyl group or an alkyl group. Therefore, generation of the diyne compound can be inhibited by addition of a substance that is oxidized in place of the compound represented by formula (1), that is, a reducing agent. The reducing agent mentioned herein is used based on the aforementioned concept.

One of specific embodiments in the case of using the compound represented by formula (1) is, for example, an embodiment in which the compound represented by formula (1) is dissolved in a solvent and a reducing agent is added to the solution (liquid phase) containing the dissolved compound.

As the reducing agent, both an inorganic reducing agent and an organic reducing agent can be used. Further, the reducing agent is preferably selected from those that functions so as to inhibit a dimerization reaction of a first ethynyl compound, in respect that generation of a diyne compound is inhibited.

Specific examples of the reducing agent include sodium sulfite, sodium thiosulfate, sodium dithionite, sodium sulfide, sodium disulfide, dithiothreitol, β-mercaptoethanol, tartaric acid or a salt thereof, ascorbic acid or a salt thereof, substituted or unsubstituted hydroxylamine or a salt thereof, and the like.

Among these compounds, an organic reducing agent is preferable, because after addition the reducing agent can be removed to a degree below the detection limit by solid-liquid separation and is more suitable for applications to a functional material such as a medical or agrochemical intermediate, liquid crystal or an electronic material. More preferable are dithiothreitol, β-mercaptoethanol, tartaric acid or a salt thereof, ascorbic acid or a salt thereof, and substituted or unsubstituted hydroxylamine or a salt thereof. Among them, tartaric acid or a salt thereof, ascorbic acid or a salt thereof are further preferable.

The amount of the reducing agent used is not particularly limited, but the reducing agent can be used in an amount of, for example, 0.001 mol to 1.0 mol relative to one mol of the compound represented by formula (1). If the amount of the reducing agent used falls in the aforementioned range, it is effective in that the reducing agent can be easily removed by liquid-liquid separation or solid-liquid separation. In particular, the amount of the reducing agent used is preferably 0.01 mol to 0.50 mol, and more preferably 0.02 mol to 0.20 mol, relative to one mol of the compound represented by formula (1).

The compound represented by the aforementioned formula (1) can be obtained by a known synthesis method. In the present invention, for example, a compound represented by the following formula (2) can be used as a raw material for the syntheses. This is one of preferred production methods in the invention.

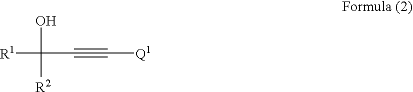

The compound represented by the aforementioned formula (2) will be hereinafter described.

In formula (2), Q¹ has the same definition as that of Q¹ in formula (1), and the preferred embodiments thereof are the same as those in formula (1). In particular, Q¹ is preferably a hydrocarbon group or a hetero atom-containing group, more preferably an aromatic hydrocarbon group or an aromatic heterocyclic group, and further preferably an aryl group. Further, an alkyl group, an aralkyl group, an alkenyl group, a heterocyclic group, and an alkynyl group are also preferable. These groups each may be unsubstituted or may have a substituent.

Moreover, Q¹ is preferably a phenyl group having at least one substituent, a naphthyl group having at least one substituent, or an anthranyl group having at least one substituent, and is more preferably a phenyl group having at least one substituent. When Q¹ in formula (2) is an aryl group (preferably, a phenyl group), examples of the substituent include a 2-carboxyl group, a 3-carboxyl group, a 3,4-dicarboxyl group, a 3,5-dicarboxyl group, a 3-amino group, a 3-acyloxy group, a 4-amino group, a 4-acylamino group, and salts thereof.

In this case, the structure of Q¹ may be changed when the compound represented by formula (2) is converted into the compound represented by formula (1). That is to say, owing to changes in the synthesis process, Q¹ in formula (1) and Q¹ in formula (2) may be different from each other.

Further, when Q¹ has plural substituents, two of the substituents may be bonded to each other. In this case, preferably, among the aforementioned substituents, plural substituents selected from the group consisting of a hydroxyl group, an amino group, an acylamino group, a carboxyl group, an acyloxy group, a sulfonyl group, and a phosphoryl group are bonded to each other to form a ring. As a preferred example, there is shown a case in which when adjacent carbon atoms are respectively substituted with a carboxyl group, these two carboxyl groups are bonded to each other to form a ring, whereby an acid anhydride is formed.

In the aforementioned formula (2), R¹ and R² each independently represent a hydrogen atom or a hydrocarbon group, and preferably, a hydrogen atom, an alkyl group having 1 to 6 carbon atoms, a cycloalkyl group having 1 to 6 carbon atoms, or an aryl group having 6 to 12 carbon atoms.

When R¹ and R² each independently represent an alkyl group having 1 to 6 carbon atoms, the alkyl group may be linear, branched or cyclic. Examples of the alkyl group include methyl, ethyl, n-propyl, isopropyl, isobutyl, t-butyl, n-hexyl and the like. Among them, methyl, ethyl and isobutyl are preferable. Further, R¹ and R2 may be bonded to each other to form a ring. Examples of a preferred ring, if the ring is formed, include a cyclopropane ring, a cyclopentane ring, a cyclohexane ring and the like.

In a case in which the structure represented by R¹ and R² is a cycloalkyl group having 1 to 6 carbon atoms, a substituted or unsubstituted alicyclic hydrocarbon group is exemplified. Specific examples of the cycloalkyl group include a cyclohexyl group, a cyclopentyl group, a cyclopropyl group, a cyclopropylmethyl group, a cyclobutyl group, and the like. Among them, a cyclohexyl group, a cyclopentyl group, and a cyclopropyl group are preferable, and a cyclohexyl group is more preferable.

When R¹ and R² each independently represent an aryl group having 6 to 12 carbon atoms, as the aryl group, a substituted or unsubstituted aromatic hydrocarbon group is exemplified. Specific examples of the aryl group include a phenyl group, a 1-naphthyl group, a 2-naphtyl group, a p-tolyl group, a 2,4,6-trimethylphenyl group (a mesityl group), and the like. Among them, a phenyl group, a 1-naphthyl group, and a p-tolyl group are preferable. A phenyl group and a p-tolyl group are more preferable, and a phenyl group is still further preferable.

Examples of the compound represented by the aforementioned formula (2) include 3-(3-hydroxy-3-methylbutyl-1-yl)aniline, dimethyl 5-(3-hydroxy-3-methylbutyl-1-yl)isophthalate, dimethyl 4-(3-hydroxy-3-methylbutyl-1-yl)phthalate, N-acetyl-4-(3-hydroxy-3-methylbutyl-1-yl)anilide, N-isobutyric acid-acetyl-4-(3-hydroxy-3-methylbutyl-1-yl)anilide, and 1-t-butoxycarbonyloxy-3-(3-hydroxy-3-methylbutyl-1-yl)benzene.

Next, a description will be given of a method of obtaining the compound represented by the aforementioned formula (1) from the compound represented by the aforementioned formula (2).

The method of obtaining the compound represented by formula (1) from the compound represented by formula (2) has been conventionally known, and this method can be achieved by causing the latter compound to react in a protic solvent under an alkali condition.

In the present invention, in order to prevent the compound of formula (1) thus generated from being changed by oxidation into the compound represented by formula (1′), a reducing agent is made to coexist in a liquid phase used in obtaining the compound represented by formula (1) before the reaction is caused.

As the reducing agent that is made to coexist in the liquid phase including the compound represented by formula (2) when the compound represented by formula (1) is generated from the compound of formula (2), inorganic and organic reducing agents both can be used as previously described. Examples thereof include sodium sulfite, sodium thiosulfate, sodium dithionite, sodium sulfide, sodium disulfide, dithiothreitol, β-mercaptoethanol, tartaric acid or a salt thereof, ascorbic acid or a salt thereof, substituted or unsubstituted hydroxylamine or a salt thereof, and the like. Among them, an organic reducing agent is more preferable, because after addition the reducing agent can be removed to a degree below the detection limit by solid-liquid separation and is more suitable for applications to a functional material such as a medical or agrochemical intermediate, liquid crystal or an electronic material. More preferable are dithiothreitol, β-mercaptoethanol, tartaric acid or a salt thereof, ascorbic acid or a salt thereof, and substituted or unsubstituted hydroxylamine or a salt thereof. Among them, tartaric acid or a salt thereof, ascorbic acid or a salt thereof are further preferable.

The amount of the reducing agent made to coexist when the compound represented by formula (1) is generated from the compound represented by formula (2) is, for example, 0.001 mol to 1.0 mol relative to one mol of the compound represented by formula (1), preferably 0.01 mol to 0.50 mol, and more preferably 0.02 mol to 0.20 mol.

When the monosubstituted ethynyl compound represented by formula (1) is produced, used or handled, a solvent may be used in order to form a liquid phase containing the monosubstituted ethynyl compound in a dissolved state. Examples of the solvent include water, toluene, n-butanol, ethanol, methanol, N-methylpyrrolidone, 1,3-dimethylimidazolidine, dimethylsulfoxide and the like.

The method of handling an ethynyl compound of the present invention is a method in which the monosubstituted ethynyl compound (first ethynyl compound) represented by the aforementioned formula (1) is handled in the presence of the reducing agent in the liquid phase in which the monosubstituted ethynyl compound is produced or used. Details and preferred embodiments of the first ethynyl compound, details and preferred embodiments of the reducing agent, and the amount of the reducing agent and its preferred range are the same as those described above.

As described above, due to the reducing agent existing in the liquid phase in which the monosubstituted ethynyl compound is handled, generation of a diyne compound is inhibited, thereby making it possible to handle the ethynyl compound while inhibiting generation of the diyne compound.

Further, in the method of using ascorbic acid or a salt thereof according to the present invention, ascorbic acid or a salt thereof is made to exist in a liquid phase including the first ethynyl compound represented by the aforementioned formula (1), so as to inhibit a dimerization reaction of the first ethynyl compound. Details and preferred embodiments of the first ethynyl compound, and the amount of ascorbic acid or a salt thereof (reducing agent) and its preferred range are the same as those described above.

The aforementioned oxidation process is inhibited by ascorbic acid or a salt thereof in place of the first ethynyl compound represented by the aforementioned formula (1), and generation of the diyne compound can be effectively inhibited.

Thus, when the monosubstituted ethynyl compound represented by the formula (1) is produced, used or handled, oxidation process caused by air, a vessel and unintended impurities can be prevented by using a reducing agent (particularly, an organic reducing agent) in a liquid phase with the monosubstituted ethynyl compound existing therein, and formation of a by-product (diyne compound) represented by the formula (1′) can be effectively prevented. As a result, a high-quality monosubstituted ethynyl compound can be stably obtained.

### EXAMPLES

The present invention will be hereinafter described more specifically with reference to the examples, but it is not limited to these examples. Note that “part(s)” and “%” are mass standards (part(s) by mass and % by mass) unless otherwise specified.

### Example 1

### Synthesis of 3-Ethynylaniline

In a 300 ml three-neck flask, 35.0 g of 3-(3-hydroxy-3-methylbutyl-1-yl) aniline (the compound represented by formula (2)), 200 g of toluene, and 1.76 g of ascorbic acid (a reducing agent) were added and stirred. Added thereto was 10.0 g of a 25% aqueous solution of sodium hydroxide, and thereafter, the temperature of the mixture was elevated to 90° C. This mixture was reacted at 90° C. for one hour, and thereafter, added thereto was an appropriate amount of 15% salt water, and cooled down to 60° C., made to stand still for 5 minutes and the aqueous phase was eliminated. Subsequently, 5% salt water was used and stirred, and made to stand still, and then, the aqueous phase was eliminated again. After concentration of the organic phase, 3-ethynyl aniline was taken out by distillation under reduced pressure (yield: 22.9 g, percent yield: 97.9%).

According to analysis using a high-speed liquid chromatograph (HPLC; hereinafter abbreviated as HPLC), 1,4-bis(3-aminophenyl)-1,3-butadiyne that is an oxidation by-product was not detected.

### Comparative Example 1

3-ethynyl aniline was synthesized without using ascorbic acid in Example 1, and was taken out from distillation under reduced pressure (yield: 20.5 g, percent yield: 87.6%).

When the distillation residue was analyzed by HPLC, 1,4-bis(3-aminophenyl)-1,3-butadiyne that is an oxidation by-product was detected. The amount thereof detected was 1.7 g (which amount corresponds to 7.3% in terms of yield).

### Example 2

### Synthesis of 5-Ethynylisophthalic Acid

In a 200 ml three-neck flask, 34.5 g of dimethyl 5-(3-hydroxy-3-methylbutyl-1-yl)isophthalate (the compound represented by the formula (2)), 60 g of water, and 2.2 g of ascorbic acid (a reducing agent) were added in a nitrogen stream, and stirred. Added thereto was 60 g of a 25% aqueous solution of sodium hydroxide, and thereafter, the mixture was heated and the temperature thereof was elevated to 80° C. The mixture was reacted at 80° C. for 4 hours, and thereafter, cooled down to 50° C. Added thereto was activated carbon, and stirred for 30 minutes. Subsequently, the activated carbon was removed by suction filtration using celite, and water used for washing a filter element was also added thereto. 50.7 g of 35.5% concentrated hydrochloric acid was added to the aforementioned aqueous solution, and the inner temperature was once elevated to 80° C., and thereafter, gradually cooled down to room temperature over 4 hours. A precipitated product was taken out by suction filtration, and washed with 50 g of running distilled water and dried by air at 50° C. for 2 days, whereby 22.6 g of white powder of 5-ethynylisophthalic acid was obtained (yield percent: 95.2%).

The white powder thus obtained was analyzed based on HPLC. The area ratio of light absorbance at 254 nm was 97.8%, and 1,4-bis(3,5-dicarboxylphenyl)-1,3-butadiyne that is an oxidation by-product was almost not detected.

### Comparative Example 2

5-ethylnylisophthalic acid was synthesized without using ascorbic acid in Example 2, and was taken out by distillation under reduced pressure (yield: 22.2 g, percent yield: 93.5%).

This product was analyzed based on HPLC, and the area ratio of the light absorbance at 254 nm was 81.1%, and 1,4-bis(3,5-dicarboxylphenyl)-1,3-butadiyne that is an oxidation by-product was detected at the area ratio of 16. 9%.

### Example 3

### Synthesis of 4-Ethynylphthalic Acid

In a 500 ml three-neck flask, 55.3 g of dimethyl 4-(3-hydroxy-3-methylbutyl-1-yl)phthalate, 300 g of water, and 3.5 of ascorbic acid were added in a nitrogen stream, and stirred. Added thereto was 80.0 g of a 25% aqueous solution of sodium hydroxide, and the temperature of the mixture was elevated, and the mixture was heated and refluxed for 10 hours. Subsequently, this mixture was cooled down to 60° C., and hydrochloric acid was added so that pH was set at 1 or less, and thereafter, cooled down gradually over 2 hours. The precipitated product thus obtained was taken out by suction filtration, and washed by 100 g of running water and dried by air at 50° C. for 2 days, whereby pale yellow powder of 4-ethynyl phthalic acid was obtained (yield: 33.6 g, percent yield: 88.3%)

The pale yellow powder thus obtained was analyzed based on HPLC. As a result, the area ratio of the light absorbance at 254 nm was 97.0% and 1,4-bis(3,4-dicarboxylphenyl)-1,3-butadiyne that is an oxidation by-product was almost not detected.

### Comparative Example 3

4-Ethynylphthalic acid was synthesized without using any ascorbic acid in Example 3, and taken out by distillation under reduced pressure (yield: 32.5 g, percent yield: 85.4%).

The synthesized product was analyzed based on HPLC. As a result, the area ratio of the light absorbance at 254 nm was 90.4%, and 1,4-bis(3,4-dicarboxylphenyl)-1,3-butadiyne that is an oxidation by-product was detected at the area ratio of 6.0%.

In the aforementioned examples, cases of producing 3-ethynylaniline, 5-ethynylisophthalic acid, and 4-ethynylphthalic acid were mainly described. However, in a case of using these compounds in a liquid phase for purposes other than production, and also in a case in which monosubstituted ethynyl compounds represented by the aforementioned formula (1) other than these compounds are produced, used or handled in a liquid phase, oxidative formation of a 1,3-diyne compound can be effectively prevented as described above.

Further, in the examples, there was shown a case of using ascorbic acid as a reducing agent, but the same effect can be obtained even in a case of using other reducing agents than ascorbic acid.

In addition, when the monosubstituted ethynyl compound represented by the aforementioned formula (1), and ascorbic acid and/or a salt thereof are mixed to form a mixed liquid, the monosubstituted ethynyl compound can be stably used in the mixed liquid without causing generation of a dimer.

What is claimed is:

1. A method of producing a first ethynyl compound represented by the following formula (1), the method comprising reacting a second ethynyl compound represented by the following formula (2) in a liquid phase in the presence of a reducing agent to obtain the first ethynyl compound: 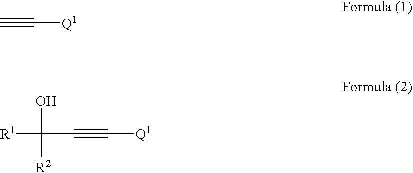
wherein Q¹ represents an organic group; R¹ and R² each independently represent a hydrogen atom or a hydrocarbon group; and R¹ and R² may be bonded to each other.

2. The method according to claim 1, wherein Q¹ is a hydrocarbon group or a hetero atom-containing group, which may have a substituent.

3. The method according to claim 2, wherein Q¹ is an aromatic hydrocarbon group or an aromatic heterocycle, which may have a substituent.

4. The method according to claim 3, wherein Q¹ is an aryl group which may have a substituent.

5. The method according to claim 3, wherein Q¹ is an alkyl group which may have a substituent, an aralkyl group which may have a substituent, an alkenyl group which may have a substituent, or an alkynyl group which may have a substituent.

6. The method according to claim 1, wherein Q¹ has at least two substituents selected from the group consisting of a hydroxyl group, an amino group, an acylamino group, a carboxyl group, an acyloxy group, a sulfonyl group and a phosphonyl group, and two adjacent substituents are bonded to each other to form a ring.

7. The method according to claim 4, wherein Q¹ is a phenyl group having at least one substituent, a naphthyl group having at least one substituent, or an anthranyl group having at least one substituent.

8. The method according to claim 4, wherein Q¹ is an aryl group having a substituent, and the substituent in Q¹ is a 2-carboxyl group, a 3-carboxyl group, a 3,4-dicarboxyl group, a 3,5-dicarboxyl group, a 3-amino group, a 3-acyloxy group, a 4-amino group, a 4-acylamino group, or a salt thereof.

9. The method according to claim 1, wherein the reducing agent functions so as to inhibit a dimerization reaction of the first ethynyl compound.

10. The method according to claim 1, wherein the reducing agent is at least one selected from the group consisting of dithiothreitol, β-mercaptoethanol, tartaric acid, ascorbic acid, substituted or unsubstituted hydroxylamine, and salts thereof.

11. The method according to claim 10, wherein the reducing agent is at least one selected from the group consisting of ascorbic acid, tartaric acid and salts thereof.

12. A method of handling an ethynyl compound, comprising handling a first ethynyl compound represented by the following formula (1) in the presence of a reducing agent in a liquid phase in which the first ethynyl compound is produced or used: 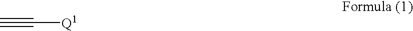
wherein Q¹ represents an organic group.

13. A method of using ascorbic acid or a salt thereof, comprising including ascorbic acid or a salt thereof in a liquid phase containing a first ethynyl compound represented by the following formula (1) to inhibit a dimerization reaction of the first ethynyl compound: 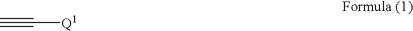
wherein Q¹ represents an organic group.
